(19) United States
(12) Patent Application Publication
(10) Pub. No.: US 2017/0129895 A1
(43) Pub. Date: May 11, 2017

(54) AMORPHOUS FORM OF BARICITINIB

(71) Applicant: SUN PHARMACEUTICAL INDUSTRIES LIMITED, Mumbai, Maharashtra (IN)

(72) Inventors: Gyanendra PANDEY, Faridabad (IN); Javeena ., South Delhi (IN); Mohan PRASAD, Gurgaon (IN); Kaptan SINGH, Gurgaon (IN); Mohan PRASAD, Gurgaon (IN)

(21) Appl. No.: 15/129,914

(22) PCT Filed: Mar. 11, 2015
(86) PCT No.: PCT/IB2015/051776

§ 371 Date: Sep. 28, 2016

(30) Foreign Application Priority Data
Mar. 28, 2014 (IN) 895/DEL/2014

(51) Int. Cl. C07D 487/04

(52) U.S. Cl. CPC C07D 487/04

(57) ABSTRACT

The present invention provides an amorphous form of baricitinib, processes for its preparation, a pharmaceutical composition comprising it, and its use for the treatment of JAK-associated diseases.

### FIELD OF THE INVENTION

The present invention provides an amorphous form of baricitinib, processes for its preparation, a pharmaceutical composition comprising it, and its use for the treatment of JAK-associated diseases.

### BACKGROUND OF THE INVENTION

Baricitinib is a Janus Kinase (JAK) inhibitor. It is chemically designated as {1-(ethylsulfonyl)-3-[4-(7H-pyrrolo[2,3-d]pyrimidin-4-yl)-1H-pyrazol-1-yl]azetidin-3-yl}acetonitrile, having the structure as depicted in Formula I.

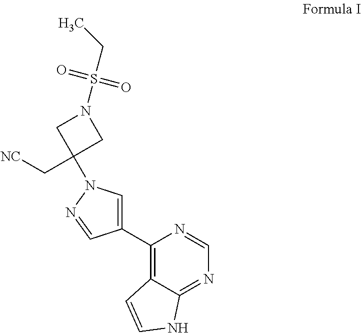 [SMILES: CCS(=O)(=O)N1CC(CC#N)(n2cc(-c3ncnc4[nH]ccc34)cn2)C1]

Processes for the preparation of baricitinib are disclosed in U.S. Pat. No. 8,158,616.

Polymorphism, the occurrence of different crystal forms, is a property of some molecules. When polymorphism occurs, the molecules arrange themselves in two or more different ways in the crystal giving rise to differences in crystal structures and physical properties such as melting point, thermal behaviors, X-ray powder diffraction (XRPD) pattern, infrared absorption fingerprint, solid state NMR spectrum, and solubility. Thus, the discovery of new polymorphic forms of a molecule is important in the development of pharmaceuticals as they may provide materials having desirable processing properties, such as ease of handling, ease of processing, storage stability, ease of purification, improved dissolution profile, and/or improved shelf-life.

There are no reported polymorphs of baricitinib.

### SUMMARY OF THE INVENTION

The present invention provides an amorphous form of baricitinib, processes for its preparation, a pharmaceutical composition comprising it, and its use for the treatment of JAK-associated diseases. The amorphous form of baricitinib is a highly pure, easy to filter, free-flowing solid. The amorphous form of baricitinib has a small average particle size and a content of residual solvents in compliance with ICH guidelines. The amorphous form of baricitinib is stable towards polymorphic conversion and exhibits good bioavailability.

A first aspect of the present invention provides an amorphous form of baricitinib.

A second aspect of the present invention provides a process for the preparation of an amorphous form of baricitinib comprising the steps of:
i) reacting 4-(1-(3-(cyanomethyl)-1-(ethylsulfonyl)azetidin-3-yl)-1H-pyrazol-4-yl)-7H-pyrrolo[2,3-d]pyrimidin-7-yl)methyl pivalate with a base in the presence of one or more solvents;
ii) completely recovering the one or more solvents from the reaction mixture;
iii) adding water; and
iv) isolating the amorphous form of baricitinib.

A third aspect of the present invention provides a process for the preparation of an amorphous form of baricitinib comprising subjecting a solution of baricitinib in a solvent to spray drying.

A fourth aspect of the present invention provides a process for the preparation of an amorphous form of baricitinib comprising subjecting a solution of baricitinib in a solvent to agitated thin film drying.

A fifth aspect of the present invention provides a process for the preparation of an amorphous form of baricitinib comprising subjecting a solution of baricitinib in a solvent to lyophilization.

A sixth aspect of the present invention provides a process for the preparation of an amorphous form of baricitinib comprising concentrating a reaction mixture containing baricitinib in a solvent under reduced pressure.

A seventh aspect of the present invention provides a pharmaceutical composition comprising an amorphous form of baricitinib and one or more pharmaceutically acceptable carriers, diluents, or excipients.

An eighth aspect of the present invention provides the use of an amorphous form of baricitinib for the treatment of JAK-associated diseases.

### BRIEF DESCRIPTION OF THE DRAWINGS

FIG. 1: X-ray powder diffraction (XRPD) pattern of an amorphous form of baricitinib.

FIG. 2: Differential Scanning calorimetry (DSC) of an amorphous form of baricitinib.

FIG. 3: Thermogravimetric Analysis (TGA) of an amorphous form of baricitinib.

FIGS. 4-7: Infra-Red (IR) spectra of an amorphous form of baricitinib.

### DETAILED DESCRIPTION OF THE INVENTION

Various embodiments and variants of the present invention are described hereinafter.

The term “JAK-associated diseases,” as used herein, includes inflammatory diseases, autoimmune disorders, diabetic nephropathy, and cancer.

The term “about,” as used herein, refers to any value which lies within the range defined by a number up to ±10% of the value.

The term “ambient temperature,” as used herein, refers to a temperature in the range of about 20° C. to about 35° C.

4-(1-(3-(Cyanomethyl)-1-(ethylsulfonyl)azetidin-3-yl)-1H-pyrazol-4-yl)-7H-pyrrolo[2,3-d]pyrimidin-7-yl)methyl pivalate can be obtained by following the process disclosed in U.S. Pat. No. 8,158,616.

The base is selected from the group consisting of inorganic and organic bases. Examples of inorganic bases include hydroxides, carbonates, and bicarbonates of alkali and alkaline earth metals. Examples of alkali and alkaline earth metal hydroxides include lithium hydroxide, sodium hydroxide, potassium hydroxide, magnesium hydroxide, calcium hydroxide, and barium hydroxide. Examples of alkali and alkaline earth metal carbonates include sodium carbonate, potassium carbonate, calcium carbonate, and magnesium carbonate. Examples of alkali metal bicarbonates include sodium bicarbonate and potassium bicarbonate. Examples of organic bases include N,N-diisopropylethylamine, triethylamine, triisopropylamine, N,N-2-trimethyl-2-propanamine, N-methylmorpholine, 4-dimethylaminopyridine, 2,6-di-tert-butyl-4-dimethylaminopyridine, 1,4-diazabicyclo[2.2.2]octane, and 1,8-diazabicyclo[5.4.0]undec-7-ene. In one embodiment of the present invention, the base used is sodium hydroxide.

The solvents are selected from the group comprising hydrocarbons, alcohols, ethers, chlorinated hydrocarbons, carboxylic acids, ketones, amides, sulphoxides, water, and mixtures thereof. Examples of hydrocarbons include benzene, toluene, and xylenes. Examples of alcohols include methanol, ethanol, 1-propanol, 1-butanol, and 2-butanol. Examples of ethers include diethyl ether, ethyl methyl ether, di-isopropyl ether, tetrahydrofuran, and 1,4-dioxane. Examples of chlorinated hydrocarbons include dichloromethane and chloroform. Examples of carboxylic acids include formic acid, acetic acid, and propionic acid. Examples of ketones include acetone, dimethyl ketone, ethyl methyl ketone, and methyl iso-butyl ketone. Examples of amides include N,N-dimethylformamide and N,N-dimethylacetamide. Examples of sulphoxides include dimethyl sulphoxide and diethyl sulphoxide. In one embodiment of the present invention, a mixture of methanol and tetrahydrofuran is used.

The preparation of the amorphous form of baricitinib may be carried out by spray drying, agitated thin film drying, lyophilization, or by concentrating a reaction mixture containing baricitinib in a solvent under reduced pressure.

In an embodiment of the present invention, the preparation of the amorphous form of baricitinib is carried out by reacting 4-(1-(3-(cyanomethyl)-1-(ethylsulfonyl)azetidin-3-yl)-1H-pyrazol-4-yl)-7H-pyrrolo[2,3-d]pyrimidin-7-yl)methyl pivalate with a base in the presence of one or more solvents at ambient temperature for about 30 minutes to about 5 hours, completely recovering the solvent(s) from the reaction mixture, adding water, and isolating the amorphous form of baricitinib.

Isolation of the amorphous form of baricitinib may be carried out by concentration, precipitation, cooling, filtration, centrifugation, or a combination thereof, followed by drying. Drying may be carried out using any suitable method such as drying under reduced pressure, air drying, or vacuum tray drying. Drying may be carried out at a temperature of about 35° C. to about 50° C. for about 10 hours to about 2 days.

In an embodiment of the present invention, the isolation of the amorphous form of baricitinib is carried out by filtration followed by drying at a temperature of about 40° C. to about 45° C. for about 24 hours.

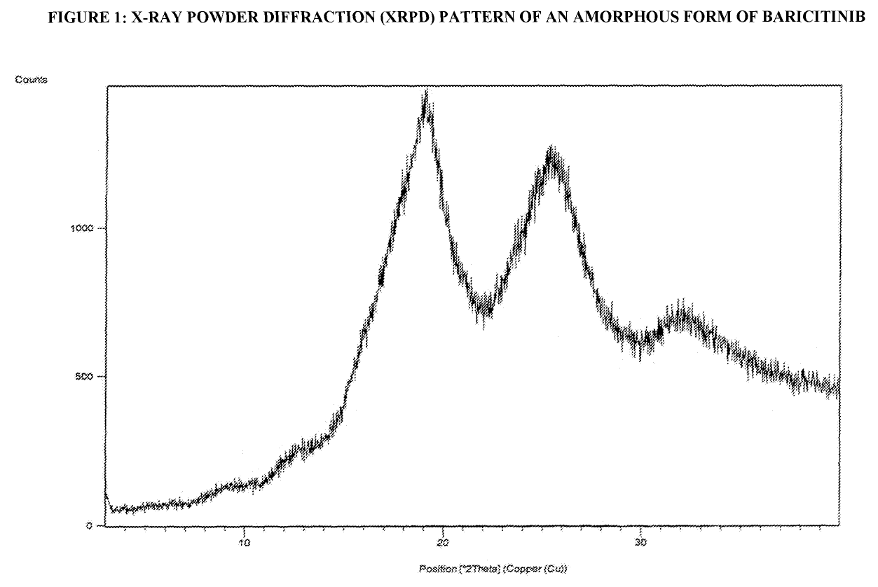

The amorphous form of baricitinib of the present invention exhibits an XRPD pattern as depicted in FIG. 1.

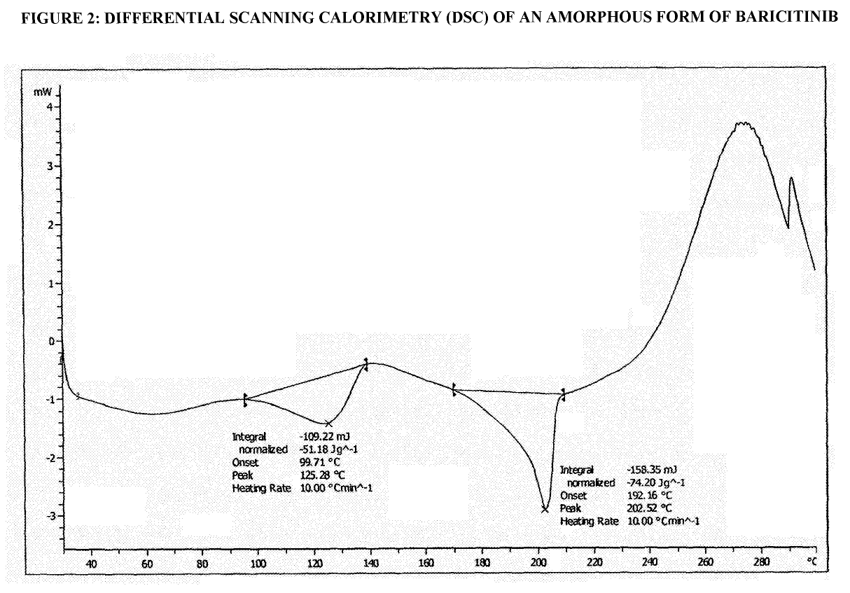

The amorphous form of baricitinib of the present invention is further characterized by a DSC thermogram having endotherms at about 125.28° C. and about 202.52° C. FIG. 2 depicts the DSC thermogram of the amorphous form of baricitinib of the present invention.

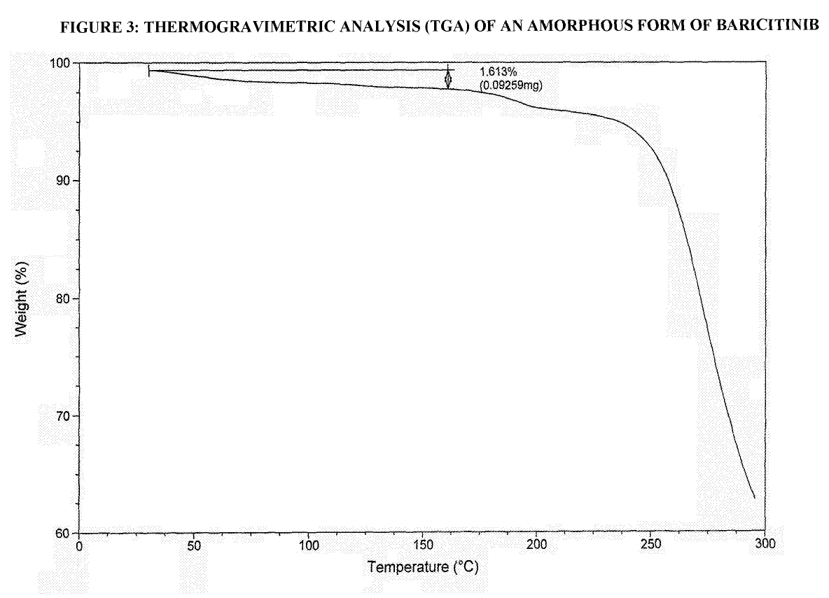

The amorphous form of baricitinib of the present invention shows a weight loss of about 1.6% as determined by TGA. FIG. 3 depicts the TGA of the amorphous form of baricitinib of the present invention.

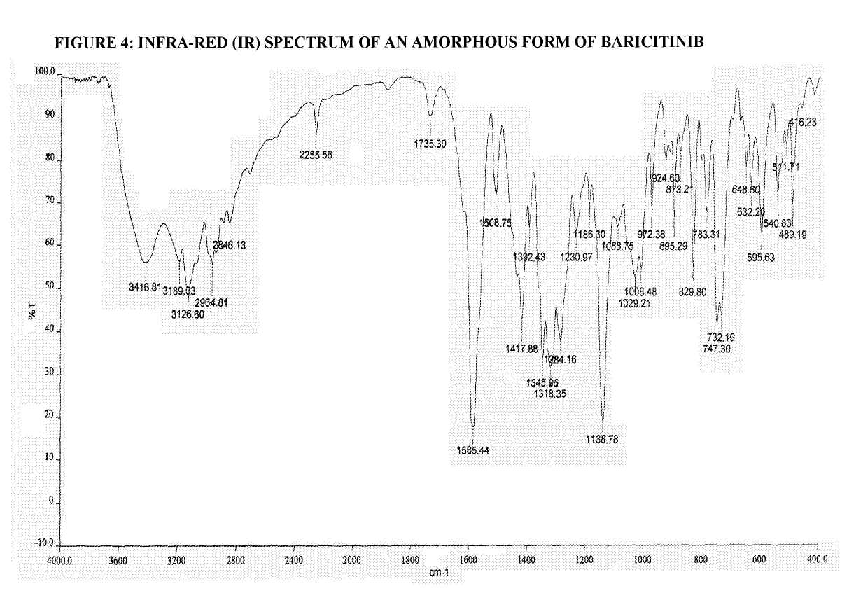

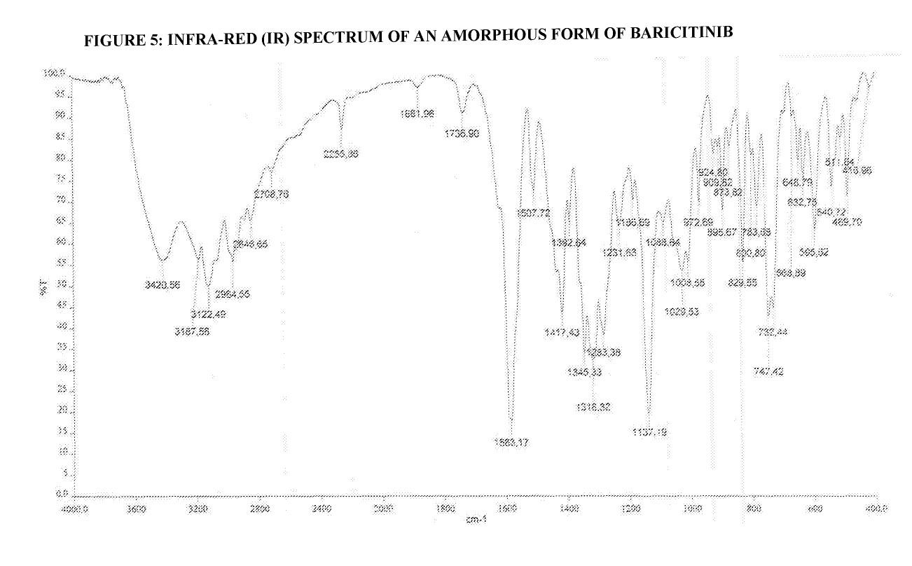

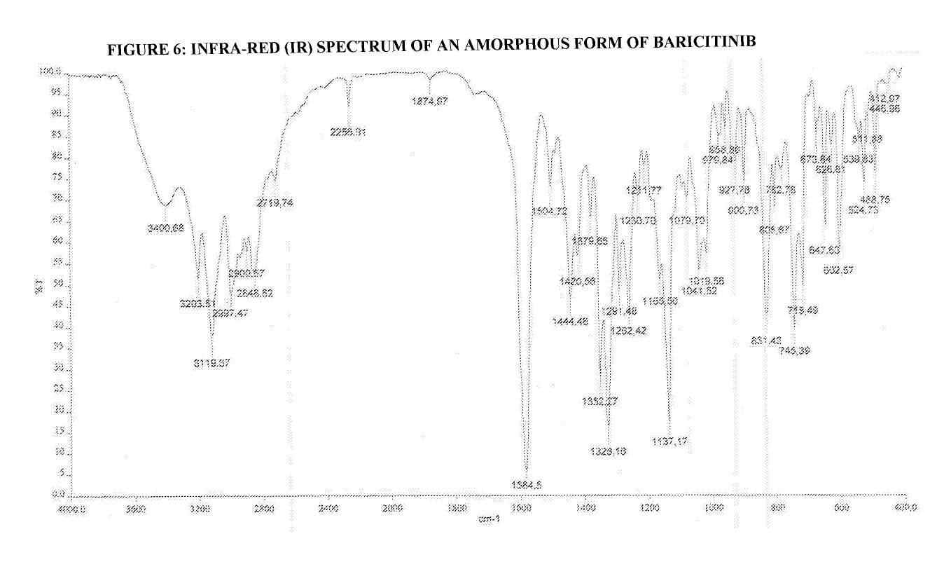

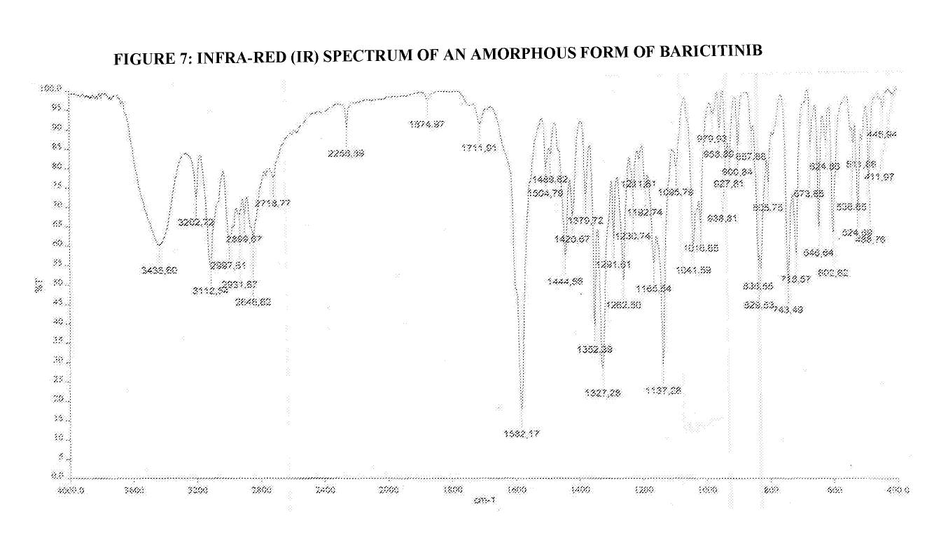

The amorphous form of baricitinib of the present invention is also characterized by an IR spectrum as depicted in FIGS. 4-7.

The amorphous form of baricitinib is a highly pure, easy to filter, free-flowing solid, having small average particle size, and a content of residual solvents in compliance with the ICH guidelines. The amorphous form of baricitinib is stable towards polymorphic conversion and has a good bioavailability.

The amorphous form of baricitinib of the present invention may be administered as part of a pharmaceutical composition for the treatment of JAK-associated diseases, including inflammatory diseases, autoimmune disorders, diabetic nephropathy, and cancer. Accordingly, in a further aspect of the present invention, there is provided a pharmaceutical composition comprising the amorphous form of baricitinib and one or more pharmaceutically acceptable carriers, diluents, or excipients, and optionally other therapeutic ingredients.

In the foregoing section, embodiments are described by way of an example to illustrate the process of the present invention. However, this is not intended in any way to limit the scope of the present invention. Several variants of the example would be evident to persons ordinarily skilled in the art which are within the scope of the present invention.

### Method

XRPD pattern was recorded using a PANalytical® Expert PRO with X'celerator® as the detector, 0.02 as step size, and 3-40° 2θ as range using CuKα radiation.

The DSC thermogram was recorded using a Mettler Toledo® DSC 821e instrument.

The TGA was recorded using a TA Instruments® Q500.

The IR spectrum was recorded using a PerkinElmer Spectrum One FT-IR spectrometer.

### EXAMPLES

### Comparative Examples

### Example 1

### Repetition of the Process According to Example 78, Method B of U.S. Pat. No. 8,158,616

4-(1-(3-(Cyanomethyl)-1-(ethylsulfonyl)azetidin-3-yl)-1H-pyrazol-4-yl)-7H-pyrrolo[2,3-d]pyrimidin-7-yl)methyl pivalate (1 g), methanol (5 mL), tetrahydrofuran (20 mL), and 1M sodium hydroxide (2.3 mL) were added into a reaction vessel at 20° C. to 25° C. The reaction mixture was stirred for 3 hours. Progress of the reaction was monitored by thin layer chromatography. On completion, the reaction mixture was quenched by adding water (20 mL). The pH was adjusted to 7.0 to 7.5 by adding IN hydrochloric acid, and the contents were stirred for 1.5 hours. No solid material was obtained.

### Example 2

### Repetition of the Process According to Example 78, Method C of U.S. Pat. No. 8,158,616

4-(1-(3-(Cyanomethyl)-1-(ethylsulfonyl)azetidin-3-yl)-1H-pyrazol-4-yl)-7H-pyrrolo[2,3-d]pyrimidin-7-yl)methyl pivalate (2 g), lithium hydroxide monohydrate (0.51 g), acetonitrile (8 mL), and 2-propanol (2 mL) were added into a reaction vessel at 20° C. to 25° C. The reaction mixture was stirred at 45° C. to 50° C. for 6 hours. Progress of the reaction was monitored by thin layer chromatography. On completion, the reaction mixture was cooled to 20° C. to 25° C. The pH was adjusted to 6.0 to 7.0 by adding 1N hydrochloric acid, and the contents were stirred overnight. No solid material was obtained.

### WORKING EXAMPLE

### Preparation of an Amorphous Form of Baricitinib

4-(1-(3-(Cyanomethyl)-1-(ethylsulfonyl)azetidin-3-yl)-1H-pyrazol-4-yl)-7H-pyrrolo[2,3-d]pyrimidin-7-yl)methyl pivalate (1 g), methanol (5 mL), tetrahydrofuran (20 mL), and 1M sodium hydroxide (2.3 mL) were added into a reaction vessel at 20° C. to 25° C. The reaction mixture was stirred for 3 hours. Progress of the reaction was monitored by thin layer chromatography. On completion, the reaction mixture was quenched by adding water (20 mL). The pH was adjusted to 7.0 to 7.5 by adding 1N hydrochloric acid, followed by completely recovering the solvent under reduced pressure at 40° C. to 50° C. A sticky material was obtained. Water (10 mL) was added to the sticky material at 20° C. to 25° C. The contents were stirred for 10 minutes. A solid material was precipitated out. The solid material was filtered, washed with water (20 mL), and then dried under reduced pressure at 40° C. to 45° C. for 24 hours to obtain the amorphous form of baricitinib.

### Yield: 81%.

The amorphous form of baricitinib may be used in a pharmaceutical composition with one or more pharmaceutically acceptable carriers, diluents, or excipients, and optionally other therapeutic ingredients. The pharmaceutical composition may be used for the treatment of JAK-associated diseases.

1. An amorphous form of baricitinib.

2. The amorphous form of baricitinib according to claim 1, characterized by an XRPD pattern substantially as depicted in FIG. 1.

3. The amorphous form of baricitinib according to claim 1, characterized by a DSC thermogram having endotherms at about 125.28° C. and about 202.52° C.

4. The amorphous form of baricitinib according to claim 1, characterized by a DSC thermogram substantially as depicted in FIG. 2.

5. The amorphous form of baricitinib according to claim 1, characterized by a TGA substantially as depicted in FIG. 3.

6. The amorphous form of baricitinib according to claim 1, characterized by an IR spectrum substantially as depicted in FIG. 4.

7. A process for the preparation of an amorphous form of baricitinib comprising:
i) reacting 4-(1-(3-(cyanomethyl)-1-(ethylsulfonyl)azetidin-3-yl)-1H-pyrazol-4- yl)-7H-pyrrolo[2,3-d]pyrimidin-7-yl)methyl pivalate with a base in the presence of one or more solvents to form a reaction mixture;
ii) completely recovering the one or more solvents from the reaction mixture;
iii) adding water; and
iv) isolating the amorphous form of baricitinib.

8. The process according to claim 7, wherein the solvent is a mixture of methanol and tetrahydrofuran.

9. The process according to claim 7, wherein the base is selected from the group consisting of inorganic and organic bases.

10. (canceled)

11. (canceled)

12. A process for the preparation of an amorphous form of baricitinib comprising, subjecting a solution of baricitinib in a solvent to spray drying, or agitated thin film drying, or lyophilization, or concentrating a reaction mixture containing baricitinib in a solvent under reduced pressure, followed by completely recovering the solvent from the reaction mixture, and isolating the amorphous form of baricitinib.

13. (canceled)

14. (canceled)

15. (canceled)

16. The process according to claim 12, wherein the solvent is selected from the group comprising of hydrocarbons, alcohols, ethers, chlorinated hydrocarbons, carboxylic acids, ketones, amides, sulphoxides, water, and mixtures thereof.

17. A pharmaceutical composition comprising an amorphous form of baricitinib according to claim 1 and one or more pharmaceutically acceptable carriers, diluents, or excipients.

18. Use of an amorphous form of baricitinib according to claim 1 for the treatment of JAK-associated diseases selected from inflammatory diseases, autoimmune disorders, diabetic nephropathy, and cancer.
